(19) United States
(12) Patent Application Publication
(10) Pub. No.: US 2017/0027469 A1
(43) Pub. Date: Feb. 2, 2017

(54) DEVICES AND METHODS FOR OBTAINING WORKABLE ECG SIGNALS USING DRY KNITTED ELECTRODES

(71) Applicant: HEALTHWATCH LTD., Herzliya (IL)

(72) Inventors: Uri AMIR, Or Yehuda (IL); Boaz SHOSHANI, Raanana (IL)

(73) Assignee: HEALTHWATCH LTD., Herzliya (IL)

(21) Appl. No.: 15/302,948

(22) PCT Filed: Apr. 16, 2015
(86) PCT No.: PCT/IL2015/050412

§ 371 Date: Oct. 7, 2016

Related U.S. Application Data
(60) Provisional application No. 61/980,595, filed on Apr. 17, 2014.

(51) Int. Cl. A61B 5/0408; A61B 5/00

(52) U.S. Cl. CPC A61B 5/04085; A61B 5/6804

(57) ABSTRACT

A garment that includes textile ECG-arm-electrodes (110LA and 110RA), such as knitted electrodes, wherein the textile ECG-arm-electrodes are located within the garment such that when the garment is worn, the textile ECG-arm-electrodes are situated adjacent to the bodily skin overlaying the outmost region of the Pectoralis major muscle, proximal to the shoulder muscle. Preferably, the positioning of the textile ECG-arm-electrodes, within the garment, is designed to match the size of the garment.

### CROSS REFERENCE TO RELATED APPLICATIONS

This application claims the benefit under 35 USC 119(e) from U.S. provisional application 61/980,595 filed on Apr. 17, 2014, the disclosure of which is included herein by reference.

This application also relates to the PCT/IL2013/050964 (964) filed on Nov. 23, 2013, the disclosure of which is included herein by reference in its entirety.

### FIELD OF THE INVENTION

The present invention relates to knitted wearable health monitoring systems and, more particularly, the present invention relates to smart garments including textile ECG arm electrodes (LA and RA), wherein the textile ECG arm electrodes are located within the garment such that when the garment is worn, the textile ECG arm electrodes are situated adjacent to the bodily skin at the outmost part of the Pectoralis major muscle, proximal to the shoulder muscle (Deltoid).

### BACKGROUND OF THE INVENTION AND PRIOR ART

In prior art monitoring garments using dry textile electrodes, there is a need to wet the electrodes in order to obtain workable/processable ECG signals, by using water or gel. Hence, prior art knitted electrodes fail to obtain clinical level ECG signal delivery from the body to the respective electrodes.

Knitted electrodes in the garment are made of conductive yarn, which conductive yarn is knitted together with other basic yarns such as Nylon, bare spandex, covered spandex and/or other types of yarn.

The positioning of an electrode on a monitored living body is of critical importance for obtaining clinical level ECG signals, especially when the monitored living body is in motion. Moreover, the repeatability of the electrodes location on the body is of critical importance for comparing ECG signals in separate measurement sessions.

In particular, prior art knitted electrodes fail to obtain high signal quality, stable and repeatable ECG and other signals, from ECG electrodes in positions RA (right arm) and LA (left arm).

Electrodes RA and LA are located at respective sides of the chest, proximal to the respective armpits, as shown in FIG. 1, which illustrates an example garment having knitted electrodes that are used to obtain 12-leads ECG signals. Due to the anatomic structure of the chest, the RA and LA electrodes are typically positioned on top of the chest muscles, in particular the Pectoralis major muscles. FIG. 2 illustrates the position of the Pectoralis major muscles 30 and the general location of the heart 20. When placing a textile electrode, such as described in '964, on top of the massive section of Pectoralis major muscles 30, the muscle acts as a capacitor that increases the impedance of the electrodes and therefor substantially reduces the amplitude and quality of the signal measured by the RA and LA electrodes 110.

There is therefore a need for a stable and repeatable positioning of the RA and LA electrodes in order to obtain high quality signals, facilitating receiving clinical level ECG, while the monitored person is either in rest or in motion.

The term “continuous monitoring”, as used herein with conjunction with a health monitoring system, refers to a health monitoring system, facilitated to monitor a living being essentially continuous, day and night, when the monitored living being is awake or asleep, and active in substantially all common activities of such living being.

The term “garment”, as used herein with conjunction with wearable clothing items, refers to seamless wearable clothing items that preferably, can be tightly worn adjacently to the body of a monitored living being, typically adjacently to the skin, including undershirts, sport shirts, brassiere, underpants, special hospital shirt, socks and the like. Typically, the term “garment” refers to a clothing item that is worn adjacently to the external surface of the user's body, under external clothing or as the only clothing, in such way that the fact that there are sensors embedded therein, is not seen by any other person in regular daily behavior. A garment article may also include a clothing article that is not an underwear article per se, but still is in direct and preferably tight contact with the skin, such as a T-shirt, sleeveless or sleeved shirts, sport-bra, tights, dancing-wear, and pants.

The term “tightly” means that specific portions of the garment, where there are electrodes or other sensors that require certain pressure on the body to obtain a satisfactory signal, are designed to be as tight as needed. However, all the other parts of the garment may not be as tight. Optionally, there is a provision to facilitate tightening or releasing certain portions of the garment, by built-in straps or other tightening means, so that the need for more or less tightness does not require the replacement of the whole garment.

The phrase “clinical level ECG”, as used herein with conjunction with ECG measurements, refers to the professionally acceptable number of leads, sensitivity and specificity needed for a definite conclusion by most cardiology physicians to suspect a risky cardiac problem (for example, arrhythmia, myocardial ischemia, heart failure) that require immediate further investigation or intervention. Currently, it is at least a 12-leads ECG and preferably 15-lead ECG, coupled with a motion/posture compensation element, and a real-time processor with adequate algorithms.

### BRIEF SUMMARY OF THE INVENTION

It is an intention of the present invention to provide devices and methods for obtaining high quality ECG signals, by providing a stable and repeatable positioning of the RA and LA electrodes, facilitating clinical level ECG, while the monitored person is either in rest or in motion, including running, jumping or walking. It is the intention of the present invention to ensure that the ECG signals are obtained from a location that is a substantially repeatable location on the monitored body.

It is an intention of the present invention to provide devices and configuration that typically positions the RA and LA electrodes against the monitored body such that the increase in electrical impedance caused by the chest muscles, in particular the Pectoralis major muscle, are minimized and do not prevent the electrodes from obtaining high quality ECG signals. The optimal positioning is at the outermost part of the Pectoralis major muscle, proximal to the shoulder muscle (Deltoid).

According to the teachings of the present invention, there is provided a garment that includes textile ECG-arm-electrodes (110LA and 110RA) such as knitted electrodes, wherein the textile ECG-arm-electrodes are located within the garment such that when the garment is worn, the textile ECG-arm-electrodes are situated adjacent to the bodily skin overlaying the outermost region of the Pectoralis major muscle, proximal to the shoulder muscle.

Preferably, the positioning of the textile ECG-arm-electrodes, within the garment, is designed to match the size of the garment.

### BRIEF DESCRIPTION OF THE DRAWINGS

The present invention will become fully understood from the detailed description given herein below and the accompanying drawings, which are given by way of illustration and example only and thus not limitative of the present invention, and wherein:

FIG. 1 (prior art) illustrates garment having knitted electrodes that are used to obtain ECG signals.

FIG. 2 illustrates a suggested positioning of the RA and LA ECG electrodes, according to some embodiment of the present invention.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein; rather, these embodiments are provided, so that this disclosure will be thorough and complete, and will fully convey the scope of the invention to those skilled in the art.

An embodiment is an example or implementation of the inventions. The various appearances of “one embodiment,” “an embodiment” or “some embodiments” do not necessarily all refer to the same embodiment. Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “one embodiment”, “an embodiment”, “some embodiments”, “another embodiment” or “other embodiments” means that a particular feature, structure, or characteristic described in connection with the embodiments is included in at least one embodiment, but not necessarily all embodiments, of the inventions. It is understood that the phraseology and terminology employed herein is not to be construed as limiting and are for descriptive purposes only.

It should be noted that orientation related descriptions such as “bottom”, “up”, “horizontal”, “vertical”, “lower”, “top” and the like, assumes that the garment is worn by a person being in a standing position.

The knitted electrodes in the garment are made of conductive yarns, wherein each conductive yarn is knitted together with other basic yarns such as Nylon, bare spandex, covered spandex and/or other types of yarn. The methods described assume usage of a Santoni knitting machine or an equivalent machine.

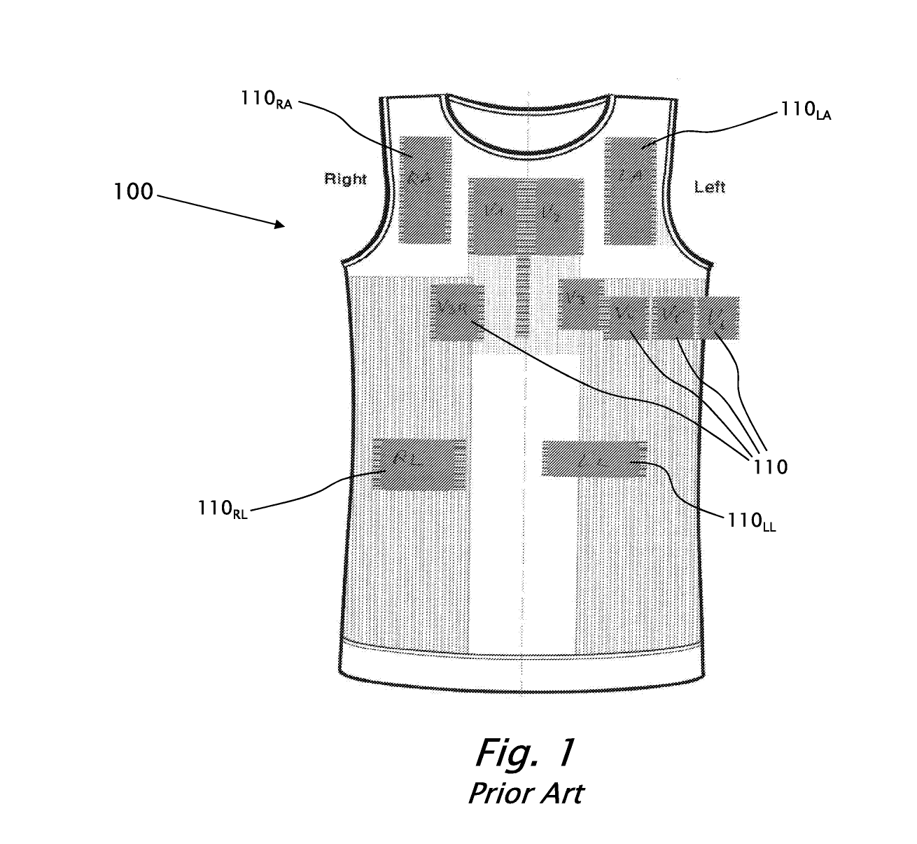

FIG. 1 illustrates a smart garment 100, such as a knitted garment, including typical 12-lead knitted electrodes 110, which electrodes are designed to be pressed against the local bodily skin surface at preconfigured bodily locations and to operate in dry conditions. Among other signals, the electrode are typically adapted to provide high quality ECG, facilitating receiving clinical level ECG, while the monitored person is either in rest or is moving, jumping or walking. In the example shown in FIG. 1, electrodes RA, LA, V1, V2, V3, V4, V5, V6, RL and LL are illustrated.

In one embodiment of the present invention, the left arm (LA) and right arm (RA) electrodes 110_{LA} and 110_{RA}, respectively, are located on the garment such that electrodes 110_{LA} and 110_{RA} are situated adjacent to the bodily skin surface that is overlaying the outmost part of the Pectoralis major muscle, proximal to the shoulder muscle (Deltoid).

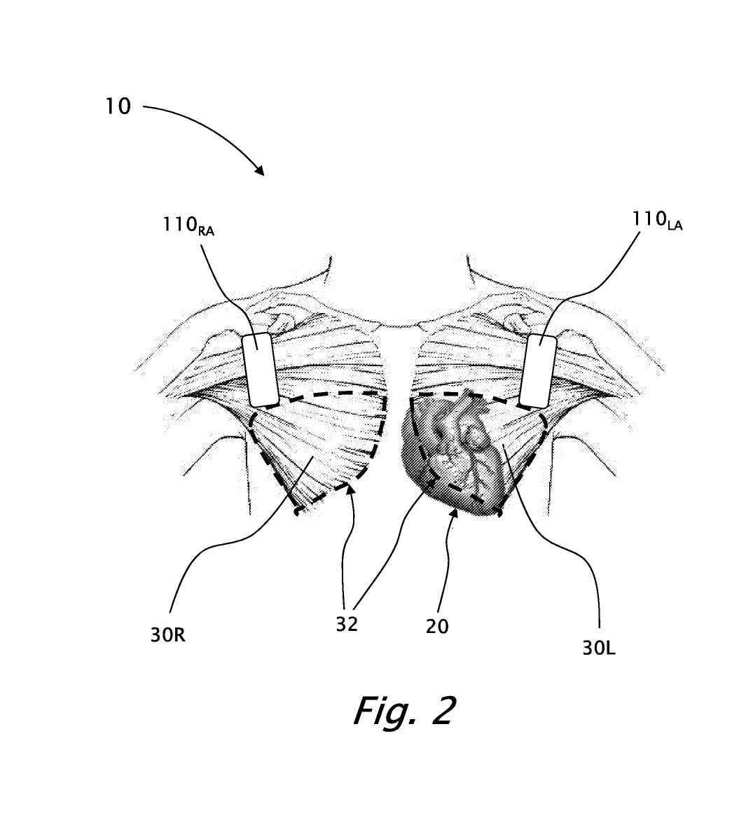

Referring back to FIG. 2, knitted electrodes 110_{LA} and 110_{RA} are shown situated above the respective massive sections 32 of the respective Pectoralis major muscles 30. Since Pectoralis major muscles 30 act as a capacitor that substantially reduces the amplitude and quality of the signal measured by electrodes 110_{LA} and 110_{RA}, the positioning of knitted electrodes 110_{LA} and 110_{RA} within the knitted monitoring garment is such that the majority area (or the whole of the area) of electrodes 110_{LA} and 110_{RA} is situated above and aside to the massive section 32 of the respective Pectoralis major muscle 30, proximal to the shoulder muscle (Deltoid). Thereby, the capacitance component of the electrical resistance of the respective knitted electrodes 110_{LA} and 110_{RA} is substantially reduced.

It should be noted that the positioning of knitted electrodes 110_{LA} and 110_{RA}, within the garment, is designed to match the size of the garment.

The invention being thus described in terms of embodiments and examples, it will be obvious that the same may be varied in many ways. Such variations are not to be regarded as a departure from the spirit and scope of the invention, and all such modifications as would be obvious to one skilled in the art are intended to be included within the scope of the claims.

What is claimed is:

1. A garment comprising textile ECG-arm-electrodes (110_{LA} and 110_{RA}), wherein said textile ECG-arm-electrodes are located within the garment such that when the garment is worn, said textile ECG-arm-electrodes are situated adjacent to the bodily skin overlaying the outmost region of the Pectoralis major muscle, proximal to the shoulder muscle.

2. A garment of claim 1, wherein said textile ECG-arm-electrodes knitted electrodes.

3. A garment of claim 1, wherein the positioning of said knitted ECG-arm-electrodes, within the garment, is designed to match the size of the garment.
